(19) United States
(12) Patent Application Publication
(10) Pub. No.: US 2012/0253032 A1
(43) Pub. Date: Oct. 4, 2012

(54) NOVEL CATIONIC LIPIDS WITH SHORT LIPID CHAINS FOR OLIGONUCLEOTIDE DELIVERY

(75) Inventors: Mark Cameron, Brick, NJ (US); Jennifer R. Davis, Richboro, PA (US); Weimin Wang, Churchville, PA (US)

(73) Assignee: Merck Sharp & Dohme Corporation

(21) Appl. No.: 13/500,733

(22) PCT Filed: Sep. 20, 2010
(86) PCT No.: PCT/US10/49420

§ 371 Date: Apr. 6, 2012

Related U.S. Application Data
(60) Provisional application No. 61/249,807, filed on Oct. 8, 2009.

(51) Int. Cl. C07J 9/00; C07J 43/00

(52) U.S. Cl. 540/113; 552/544

(57) ABSTRACT

The instant invention provides for novel cationic lipids with short lipid chains that can be used in combination with other lipid components such as cholesterol and PEG-lipids to form lipid nanoparticles with oligonucleotides, to facilitate the cellular uptake and endosomal escape, and to knockdown target mRNA both in vitro and in vivo while decreasing inflammatory toxicities.

### BACKGROUND OF THE INVENTION

The present invention relates to novel cationic lipids with short lipid chains that can be used in combination with other lipid components such as cholesterol and PEG-lipids to form lipid nanoparticies with oligonucleotides, to facilitate the cellular uptake and endosomal escape, and to knockdown target mRNA both in vitro and in vivo.

Cationic lipids and the use of cationic lipids in lipid nanoparticles for the delivery of oligonucleotides, in particular siRNA and miRNA, have been previously disclosed. (See US patent applications: US 2006/0240554 and US 2008/0020058). Lipid nanoparticles and use of lipid nanoparticles for the delivery of oligonucleotides, in particular siRNA and miRNA, has been previously disclosed. (See US patent applications: US 2006/0240554 and US 2008/0020058). Oligonucleotides (including siRNA and miRNA) and the synthesis of oligonucleotides has been previously disclosed. (See US patent applications: US 2006/0240554 and US 2008/0020058).

A major liability of lipid nanoparticles is their potential to cause inflammatory toxicities through activation of the innate immune response. This inflammatory response leads to tissue infiltration of monocytes and neutrophils, which ultimately causes tissue necrosis, hypotension, and other potentially severe sepsis-like toxicities. Abrams et al., Molecular Therapy (advance online publication 8 Sep. 2009. doi:10.1038/mt.2009.208).

It is an object of the instant invention to provide novel cationic lipids with short lipid chains that can be used in combination with other lipid components such as cholesterol and PEG-lipids to form lipid nanoparticles with oligonucleotides, to facilitate the cellular uptake and endosomal escape, and to knockdown target mRNA both in vitro and in vivo while decreasing inflammatory toxicities.

### SUMMARY OF THE INVENTION

The instant invention provides for novel cationic lipids with short lipid chains that can be used in combination with other lipid components such as cholesterol and PEG-lipids to form lipid nanoparticles with oligonucleotides, to facilitate the cellular uptake and endosomal escape, and to knockdown target mRNA both in vitro and in vivo while decreasing inflammatory toxicities.

### BRIEF DESCRIPTION OF THE FIGURES

FIG. 1: Mouse in vivo Cytokine IL-6 Induction 3 hour post injection.

FIG. 2: Mouse in vivo Cytokine mKC Induction 3 hour post injection.

### DETAILED DESCRIPTION OF THE INVENTION

The various aspects and embodiments of the invention are directed to the utility of novel cationic lipids with short lipid chains useful in lipid nanoparticles to deliver oligonucleotides, in particular, siRNA and miRNA, to any target gene. (See US patent applications: US 2006/0240554 and US 200810020058). The cationic lipids of the instant invention, are useful components in a lipid nanoparticle for the delivery of oligonucleotides, specifically siRNA and miRNA.

In a first embodiment of this invention, the cationic lipids are illustrated by the Formula A:

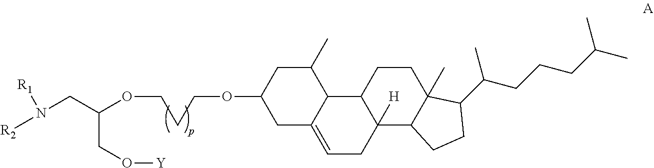

wherein:

p is 1 to 8;

R¹ and R² are independently selected from H, (C₁-C₁₀)alkyl, heterocyclyl, and a polyamine, wherein said heterocyclyl or polyamine is optionally substituted with one to three substituents selected from R³, or R¹ and R² can be taken together with the nitrogen to which they are attached to form a monocyclic heterocycle with 4-7 members optionally containing, in addition to the nitrogen, one or two additional heteroatoms selected from N, O and S, said monocylcic heterocycle optionally substituted with one to three substituents selected from R³;

R³ is independently selected from: halogen, OR⁴, SR⁴, CN, CO₂R⁴, CON(R⁴)₂;

R⁴ is independently selected from: H, (C₁-C₁₀)alkyl and aryl; and

Y is a (C₄-C₈)alkyl, (C₄-C₈)perfluoroalkyl, or a (C₄-C₈)alkenyl;

or any pharmaceutically acceptable salt or stereoisomer thereof.

In another embodiment, the invention features a compound having Formula A, wherein:

p is 1 to 8;

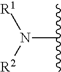

is selected from:

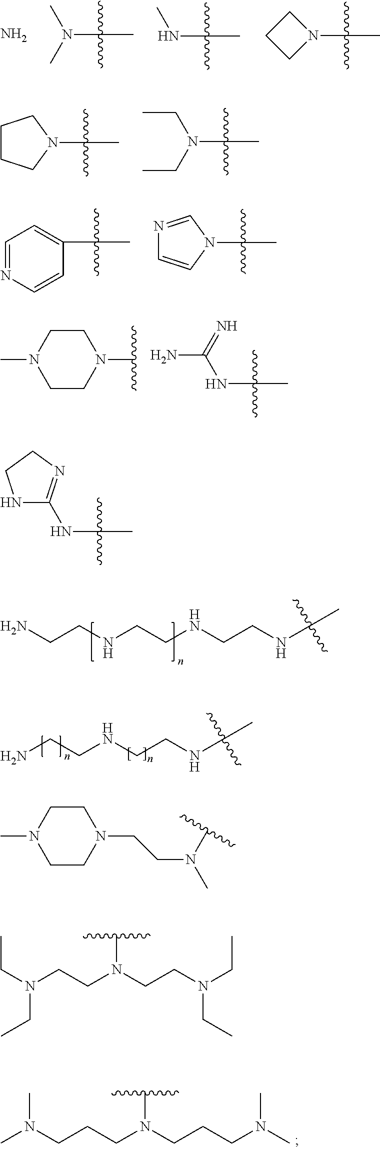

n is 1 to 10; and

Y is a (C₄-C₈)alkyl, (C₄-C₈)perfluoroalkyl, or a (C₄-C₈)alkenyl; or any pharmaceutically acceptable salt or stereoisomer thereof.

Specific cationic lipids are:
(2S)-2-{4-[(3β)-cholest-5-en-3-yloxy]butoxy}-N,N-dimethyl-3-(octyloxy)propan-1-amine (Compound 4);
(2R)-2-{4-[(3β)-cholest-5-en-3-yloxy]butoxy}-N,N-dimethyl-3-(octyloxy)propan-1-amine (Compound 5);
(2R)-2-({6-[(3β)-cholest-5-en-3-yloxy]hexyl}oxy)-N,N-dimethyl-3-(octyloxy)propan-1-amine (Compound 6);
(2R)-2-({8-[(3β)-cholest-5-en-3-yloxy]octyl}oxy)-N,N-dimethyl-3-(octyloxy)propan-1-amine (Compound 7);
1-[(2R)-2-({8-[(3β)-cholest-5-en-3-yloxy]octyl}oxy)-3-(octyloxy)propyl]guanidine (Compound 8);
1-[(2R)-2-{4-[(3β)-cholest-5-en-3-yloxy]butoxy}-3-(octyloxy)propyl]guanidine (Compound 9);
(3β)-3-(4-{[(2R)-1-(octyloxy)-3-(pyrrolidinyl-1-yl)propan-2-yl]oxy}butoxy)cholest-5-ene (Compound 10); and
(3β)-3-[(8-{[(2S)-1-(octyloxy)-3-(pyrrolidinyl-1-yl)propan-2-yl]oxy}octyl)oxy]cholest-5-ene (Compound 11);
   or any pharmaceutically acceptable salt or stereoisomer thereof.

In another embodiment, the cationic lipids disclosed are useful for the preparation of lipid nanoparticles.

In another embodiment, the cationic lipids disclosed are useful components in a lipid nanoparticle for the delivery of oligonucleotides.

In another embodiment, the cationic lipids disclosed are useful components in a lipid nanoparticle for the delivery of siRNA and miRNA.

In another embodiment, the cationic lipids disclosed are useful components in a lipid nanoparticle for the delivery of siRNA.

The cationic lipids of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the cationic lipids disclosed herein may exist as tautomers and both tautomeric forms are intended to be encompassed by the scope of the invention, even though only one tautomeric structure is depicted.

When any variable (e.g. R³) occurs more than one time in any constituent, its definition on each occurrence is independent at every other occurrence. Also, combinations, of substituents and variables are permissible only if such combinations result in stable compounds. If the ring system is bicyclic, it is intended that the bond be attached to any of the suitable atoms on either ring of the bicyclic moiety.

It is understood that substituents and substitution patterns on the cationic lipids of the instant invention can be selected by one of ordinary skill in the art to provide cationic lipids that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

It is understood that one or more Si atoms can be incorporated into the cationic lipids of the instant invention by one of ordinary skill in the art to provide cationic lipids that are chemically stable and that can be readily synthesized by techniques known in the art from readily available starting materials.

In the compounds of Formula A, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of Formula A. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within Formula A can be prepared without undue experimentation by conventional techniques well known to those skilled in the art or by processes analogous to those described in the Scheme and Examples herein using appropriate isotopically-enriched reagents and/or intermediates.

As used herein, “alkyl” means a saturated aliphatic hydrocarbon having the specified number of carbon atoms.

As used herein, “alkenyl” means an unsaturated aliphatic hydrocarbon having the specified number of carbon atoms.

As used herein, “aryl” is intended to mean any stable monocyclic or bicyclic carbon ring of up to 7 atoms in each ring, wherein at least flexing is aromatic. Examples of such aryl elements include phenyl, naphthyl, tetrahydro-naphthyl, indanyl and biphenyl.

As used herein, “heterocyclyl” means a 4- to 10-membered aromatic or nonaromatic heterocycle containing from 1 to 4 heteroatoms selected from the group consisting of O, N and S, and includes bicyclic groups. “Heterocyclyl” therefore includes, the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl; quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof all of which are optionally substituted with one to three substituents selected from R³.

As used herein, “polyamine” means compounds having two or more amino groups. Examples include putrescine, cadaverine, spermidine, and spermine.

In an embodiment,

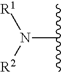

is selected from:

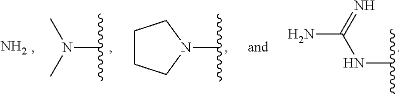

In an embodiment, n is 1 to 5.

In an embodiment, p is 1 to 8.

In an embodiment, R³ is selected from: halogen, OR⁴, SR⁴, CN, CO₂R⁴, CON(R⁴)₂.

In an embodiment, R⁴ is selected from: H, (C₁-C₆)alkyl and phenyl.

In an embodiment, Y is a (C₄-C₈)alkyl, (C₄-C₈)perfluoroalkyl, or a (C₄-C₈)alkenyl.

In an embodiment, Y is a (C₈)alkyl.

Included in the instant invention is the free form of cationic lipids of Formula A, as well as the pharmaceutically acceptable salts and stereoisomers thereof. Some of the isolated specific cationic lipids exemplified herein are the protonated salts of amine cationic lipids. The term “free form” refers to the amine cationic lipids in non-salt form. The encompassed pharmaceutically acceptable salts not only include the isolated salts exemplified for the specific cationic lipids described herein, but also all the typical pharmaceutically acceptable salts of the free form of cationic lipids of Formula A. The free form of the specific salt cationic lipids described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant cationic lipids can be synthesized from the cationic lipids of this invention which contain a basic or acidic moiety by conventional chemical methods. Generally, the salts of the basic cationic lipids are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the cationic lipids of this invention include the conventional non-toxic salts of the cationic lipids of this invention as formed by reacting a basic instant cationic lipids with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic (TFA) and the like.

When the cationic lipids of the present invention are acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glutamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the cationic lipids of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

### EXAMPLES

Examples provided are intended to assist in a further understanding of the invention. Particular materials employed, species and conditions are intended to be further illustrative of the invention and not limitative of the reasonable scope thereof. The reagents utilized in synthesizing the cationic lipids are either commercially available or are readily prepared by one of ordinary skill in the art.

The following lipid nanoparticle compositions (LNPs) of the instant invention are useful for the delivery of oligonucleotides, specifically siRNA and miRNA:

### Cationic Lipid/Cholesterol/PEG-DMG 56.6/38/5.4;

### Cationic Lipid/Cholesterol/PEG-DMG 60/38/2;

### Cationic Lipid/Cholesterol/PEG-DMG 67.3/29/3.7;

### Cationic Lipid/Cholesterol/PEG-DMG-49.3/47/3.7; and

### Cationic Lipid/Cholesterol/PEG-DMG 50.3/44.3/5.4.

Synthesis of the novel cationic lipids is a convergent process that finalizes with the alkylation of the amino alcohol (i) by the mesylate (ii) to afford the requisite cationic lipid (iii).

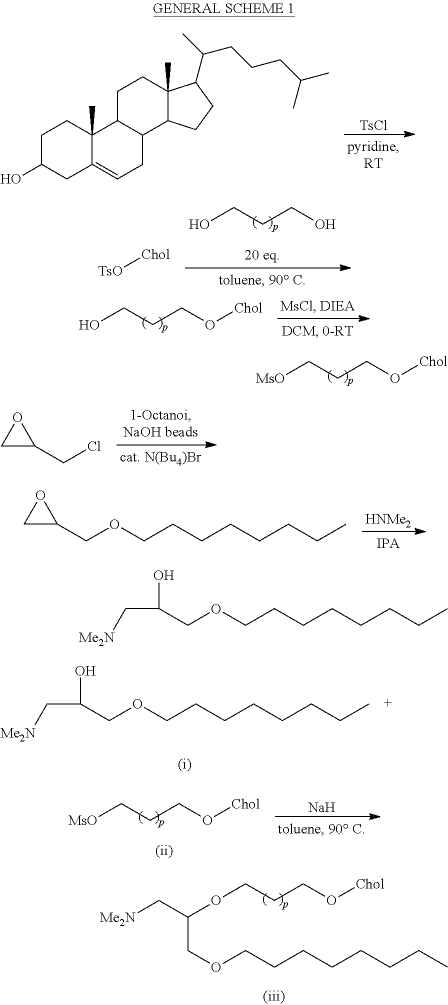

### Preparation of (2S)-2-[(octyloxy)methyl]oxirane (Compound 1)

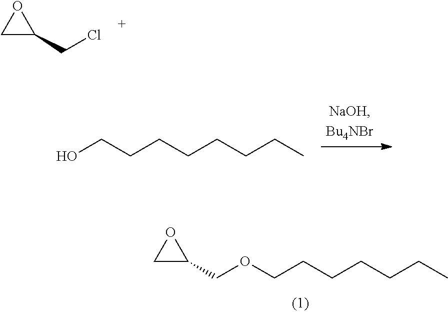

To a stirred, cooled (5° C.), mixture of 1-octanol (238.0 mL, 1.487 mmol) and Bu₄NBr (24.1 g, 74.9 mmol) was added R-epichlorohydrin (249.0 g, 2.695 mmol) in one portion. The mixture stirred at 5° C. for several hours, and warmed slowly to room temperature overnight. The reaction mixture was partitioned between hexanes (18′75 mL) and saturated NaHCO₃ solution (625 mL). The organic layer collected and washed with water (2×625 mL) and then washed with brine (625 mL). The organic solution was collected and volatiles evaporated under reduced pressure to give crude product (1, 299.9 g). Purification through chromatography afforded product (236.0 g) in 85% yield. ¹H NMR (400 MHz, CDCl₃) δ 3.70 (1H, dd, J=3.1, 12.1 Hz); 3.52-3.45 (2H, complex); 3.38 (1H, dd, J=5.5, 11.5 Hz); 3.15 (1H, complex); 2.80 (1H, dd, J=4.2, 5.0 Hz); (1H, dd, J=2.7, 5.1 Hz); 1.60-1.56 (2H, complex); 1.36-1.27 (10H, complex); 0.88 (3H, t, J=7.2 Hz) ppm.

The product of this reaction was first assigned the 2R stereochemistry, which would be obtained via a SN2 mechanism at the carbon bearing the halide atom with no change at the asymmetric carbon centre.

However, subsequent studies (experiments on the reaction of lineolyl alcohol with S-epichlorohydrin), under conditions described, Lewis acid conditions and in conjunction with vibrational circular dichroism (VCD), a valid spectroscopic method for determining absolute configuration of chiral molecules (R. K. Dukor and L. A. Nafie, in Encyclopedia of Analytical Chemistry: Instrumentation and Applications, Ed. R. A. Meyers (Wiley, Chichester, 2000) 662-676.) have revealed the reaction occurred via a SN2′ mechanism ie reaction at the terminal carbon of the epoxide leading to subsequent ring opening, followed by an in situ ring closing step that leads to inversion of stereochemistry at the asymmetric carbon.

As a result of these studies, the product from this reaction was reassigned the 2S stereochemistry: (2S)-2-[(octyloxy)methyl]oxirane.

### Preparation of (2S)-1-(dimethylamino)-3-(octyloxy)propan-2-ol (Compound 2)

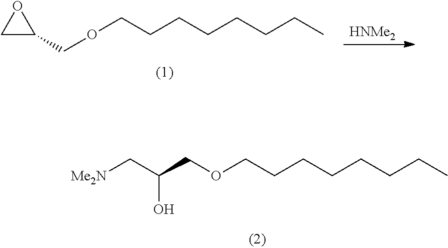

A solution of the epoxide (1, 10.00 g, 53.7 mmol) in 2M dimethylamine in methanol (400 mL, 800 mmol) was stirred overnight at ambient temperature. Volatiles were evaporated under reduced pressure to afford product (2, 11.30 g) in 91% yield. ¹H NMR (400 MHz, CDCl₃) δ 3.73 (1H, m); 3.44-3.26 (4H, complex); 2.32 (1H, dd J=9.6, 12.0 Hz); 2.20 (6H, s); 2.15 (1H, dd, J=4.0, 11.6 Hz); 1.50-1.42 (2H, complex); 1.25-1.18 (10H, complex); 0.78 (3H, t, J=7.2 Hz) ppm.

### Preparation of (2S)-2-{4-[(3β)-cholest-5-en-3-yloxy]butoxy}-N,N-dimethyl-3-(octyloxy)propan-1-amine (Compound 4)

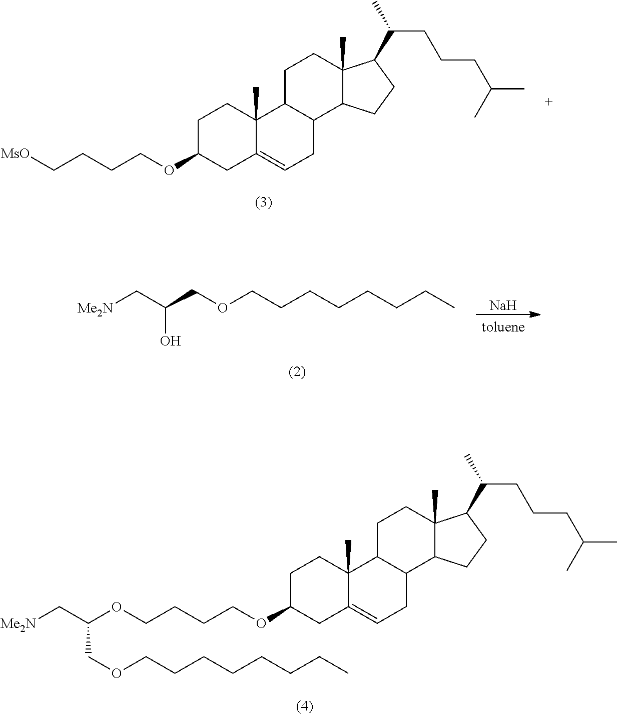

To a stirred solution of the amino alcohol (2, 7.00 g, 30.3 mmol) in dry toluene (35 mL) was added sodium hydride (2.18 g, 91.0 mmol). The mixture stirred for 30 minutes and mesylate (3, 17.04 g, 31.8 mmol) added. The mixture was then heated at 70° C. overnight. The reaction mixture cooled to 15-20° C., isopropyl alcohol (20 mL) cautiously added, and stirred for 30 minutes. The mixture was partitioned between hexanes (200 mL) and 10% aqueous Na₂CO₃ (200 mL). The organic solution collected, volatiles evaporated and crude purified through silica gel chromatography to afford product (4, 9.3 g, 13.8 mmol) in 46% yield. C₄₄H₈₁NO₃: HRMS (ESI positive) M+H, theory m/z 672.6289, measured m/z 672.6313 amu. ¹H NMR (400 MHz, CDCl₃) δ 5.34 (1H, m); 3.59 (1H, m); 3.48-3.39 (8H, complex); 3.12 (1H, m); 2.40-2.30 (3H complex); 2.25 (6H, s); 2.15 (1H, br t); 2.03-0.84 (57H, complex); 0.67 (s, 3H) ppm.

Compounds 541 are novel cationic lipids, example 12 is S-Octyl CLinDMA. The compounds below can be prepared according to the Scheme above utilizing the appropriate enantiomer of epichlorohydrin.

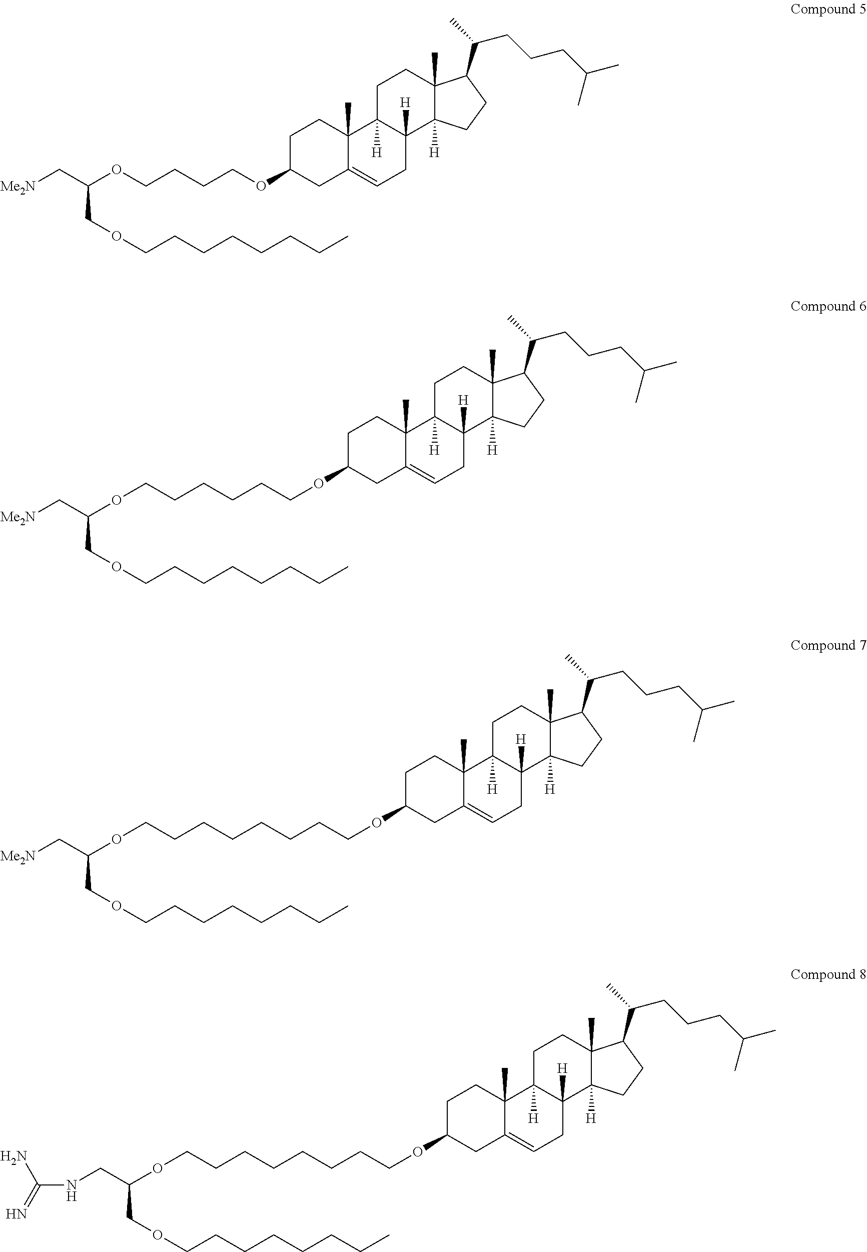

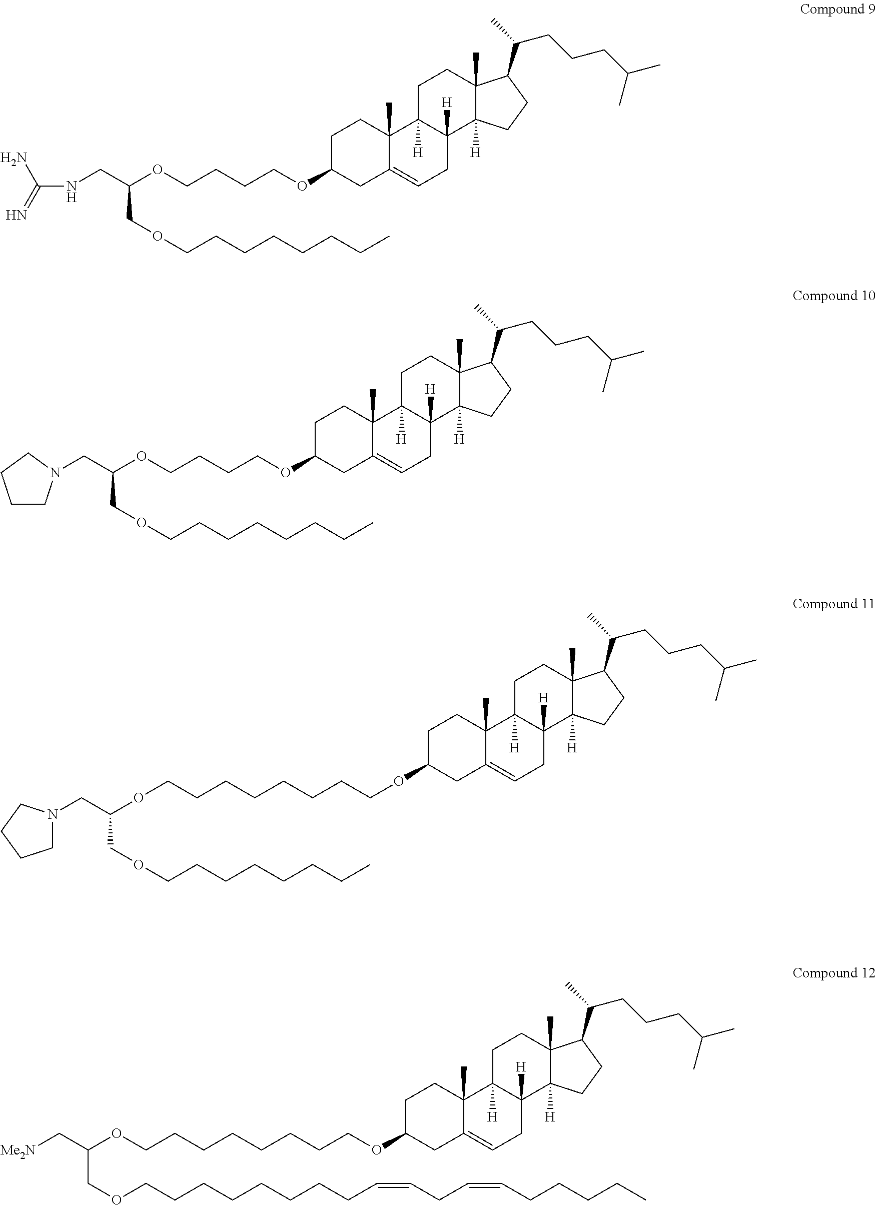

### Compound 5 (2R)-2-{4-[(3β)-cholest-5-en-3-yloxy]butoxy}-N,N-dimethyl-3-(octyloxy)propan-1-amine

2.16 g, 3.21 mmol, 50% yield. C₄₄H₈₁NO₃: HRMS (ESI positive) M+H, theory m/z 672.6289, measured m/z 672.6264 amu. ¹H NMR (400 MHz, CDCl₃) δ 5.34 (1H, m); 3.59 (1H, m); 3.45 (8H, m); 3.12 (1H, m); 2.40-2.30 (3H, m); 2.25 (6H, s); 2.10 (1H, m); 2.00-0.83 (57H, complex); 0.67 (s, 3H) ppm.

### Compound 6 (2R)-2-({6-[(3β)-cholest-5-en-3-yloxy]hexyl}oxy)-N,N-dimethyl-3-(octyloxy)propan-1-amine

2.76 g, 3.94 mmol, 61% yield. C₄₆H₈₅NO₃: HRMS (ESI positive) M+H, theory m/z 700.6602, measured m/z 700.6597 amu. ¹H NMR (400 MHz, CDCl₃) δ 5.34 (1H, m); 3.60-3.09 (9H, m); 3.12 (1H, m); 2.41-2.33 (3H, m); 2.25 (6H, s); 2.18 (1H, m); 2.15-0182 (61H, complex); 0.68 (3H, s) ppm.

### Compound 7 (2R)-2-({8-[(3β)-cholest-5-en-3-yloxy]octyl}oxy)-N,N-dimethyl-3-(octyloxy)propan-1-amine

1.60 g, 2.12 mmol, 57% yield. C₄₈H₈₉NO₃: HRMS (ESI positive) M+H, theory m/z 728.6915, measured m/z 728.6910 amu. ¹H NMR (400 MHz, CDCl₃) δ 5.34 (1H, m); 3.61-3.45 (9H, m) 112 (1H, m); 2.43-2.33 (3H, m); 2.26 (6H, s); 2.19 (1H, m); 2.05-0.83 (65H, complex); 0.68 (3H, s) ppm.

### Compound 8 1-[(2R)-2-({8-[(3β)-cholest-5-en-3-yloxy]octyl}oxy)-3-(octyloxy)propyl]guanidine

2.96 g, 4.0 mmol, 82% yield. C₄₇H₈₇N₃O₃: HRMS (ESI positive) M+H, theory m/z 742.6826, measured m/z 742.6820 amu. ¹H NMR (500 MHz, CDCl₃) δ 7.92 (1H, t, J=6.5 Hz); 7.48 (2H, br); 7.20 (1H, br); 5.34 (1H, complex); 3.57 (1H, multiplet); 3.51-3.31 (7H, complex); 3.28 (1H, multiplet); 3.12 (1H, multiplet); 2.36 (1H, br d); 2.18 (1H, br t); 2.03-1.79 (8H, complex); 1.60-0.86 (59H, complex); 0.68 (3H, s) ppm.

### Compound 9 1-[(2R)-2-{4-[(3β)-cholest-5-en-3-yloxy]butoxy}-3-(octyloxy)propyl]guanidine

1.93 g, 2.8 mmol, 84% yield. C₄₃H₇₉N₃O₃: HRMS (ESI positive) M+H, theory m/z 686.6194, measured m/z 686.6212 amu. ¹H NMR (500 MHz, CDCl₃) δ 7.96 (1H, t, J=6.5 Hz); 7.48 (2H, br); 7.10 (1H, br); 5.34 (1H, complex); 3.62 (1H, multiplet); 3.56-3.36 (7H, complex); 3.27 (1H, multiplet); 3.12 (1H, multiplet); 2.35 (1H, br d); 2.17 (1H, br t); 2.02-1.79 (8H, complex); 1.65-0.86 (51H, complex); 0.68 (3H, s) ppm.

### Compound 10 (3β)-3-(4-{[(2R)-1-(octyloxy)-3-(pyrrolidinyl-1-yl)propan-2-yl]oxy}butoxy)cholest-5-ene

2.48 g, 3.6 mmol, 66% yield. C₄₆H₈₃NO₃: HRMS (ESI positive) M+H, theory m/z 698.6446, measured m/z 698.6462 amu. ¹H NMR (500 MHz, CDCl₃) δ 5.35 (1H, complex); 3.63 (1H, multiplet); 3.56-3.44 (8H, complex); 3.13 (1H, complex); 2.65 (1H, br d); 2.55-2.47 (6H, complex); 2.35 (1H, br d); 2.18 (1H, br t); 2.05-0.87 (60H, complex); 0.68 (3H, s) ppm.

### Compound 11 (3β)-3-[(8-{[(2S)-1-(octyloxy)-3-(pyrrolidinyl-1-yl)propan-2-yl]oxy}octyl)oxy]cholest-5-ene

2.90 g, 3.9 mmol, 76% yield. C₅₀H₉₁NO₃: HRMS (ESI positive) M+H, theory m/z 754.7072, measured m/z 754.7058 amu. ¹H NMR (500 MHz, CDCl₃) δ 5.34 (1H, complex); 3.61-3.40 (9H, complex); 3.12 (1H, complex); 2.65 (1H, br d); 2.55-2.46 (6H, complex); 2.35 (1H, br d); 2.18 (1H, br t); 2.05-1.79 (8H, complex); 1.65-0.86 (60H, complex); 0.68 (3H, s) ppm.

### LNP Compositions

### LNP Process Description:

The Lipid Nano-Particles (LNP) are prepared by an impinging jet process. The particles are formed by mixing-equal volumes of lipids dissolved in alcohol with siRNA dissolved in a citrate buffer. The lipid solution contains a novel cationic lipid of the instant invention, a helper lipid (cholesterol) and PEG (PEG-DMG) lipid at a concentration of 5-15 mg/mL with a target of 9-12 mg/mL in an alcohol (for example ethanol). The ratio of the lipids has a mole percent range of 25-98 for the cationic lipid with a target of 45-65, the helper lipid has a mole percent range from 0-75 with a target of 30-50 and the PEG lipid has a mole percent range from 1-6 with a target of 2-5. The siRNA solution contains one or more siRNA sequences at a concentration range from 0.7 to 1.0 mg/mL with a target of 0.8-0.9 mg/mL in a sodium citrate: sodium chloride buffer pH 4. The two liquids are mixed in an impinging jet mixer instantly fowling the LNP. The teeID has a range from 0.25 to 1.0 mm and a total flow rate from 10-200 mL/min. The combination of flow rate and tubing ID has effect of controlling the particle size of the LNPs between 50 and 200 nm. The mixed LNPs are held from 30 minutes to 48 hrs prior to a dilution step. The dilution step comprises similar impinging jet mixing which instantly dilutes the LNP. This process uses tubing IDs ranging from 1 mm ID to 5 mm ID and a flow rate from 10 to 400 mL/min. The LNPs are concentrated and diafiltered via an ultrafiltration process where the alcohol is removed and the citrate buffer is exchanged for the final buffer solution such as phosphate buffered saline. The ultrafiltration process uses a tangential flow filtration format (TFF). This process uses a membrane nominal molecular weight cutoff range from 30-500 KD. The membrane format can be hollow fiber or flat sheet cassette. The TFF processes with the proper molecular weight cutoff retains the LNP in the retentate and the filtrate or permeate contains the alcohol; citrate buffer; final buffer wastes. The TFF process is a multiple step process with an initial concentration to a siRNA concentration of 1-3 mg/mL. Following concentration, the LNPs solution is diafiltered against the final buffer for 15-20 volumes to remove the alcohol and perform buffer exchange. The material is then concentrated an additional 1-3 fold. The final steps of the LNP process are to sterile filter the concentrated LNP solution and vial the product.

### Analytical Procedure:

1) siRNA Concentration

The siRNA duplex concentrations are determined by Strong Anion-Exchange High-Performance Liquid Chromatography (SAX-HPLC) using Waters 2695 Alliance system (Water Corporation, Milford Mass.) with a 2996 PDA detector. The LNPs, otherwise referred to as RNAi Delivery Vehicles (RDVs), are treated with 0.5% Triton X-100 to free total siRNA and analyzed by SAX separation using a Dionex BioLC DNAPac PA 200 (4×250 mm) column with UV detection at 254 nm. Mobile phase is composed of A: 25 mM NaClO₄, 10 mM Tris, 20% EtOH, pH 7.0 and B: 250 mM NaClO₄, 10 mM Tris, 20% EtOH, pH 7.0 with liner gradient from 0-15 min and flow rate of 1 ml/min. The siRNA amount is determined by comparing to the siRNA standard curve,

### 2) Encapsulation Rate

Fluorescence reagent SYBR Gold is employed for RNA quantitation to monitor the encapsulation rate of RDVs. RDVs with or without Triton X-100 are used to determine the free siRNA and total siRNA amount. The assay is performed using a SpectraMax M5e microplate spectrophotometer from Molecular Devices (Sunnyvale, Calif.). Samples are excited at 485 nm and fluorescence emission was measured at 530 nm. The siRNA amount is determined by comparing to the siRNA standard curve.

Encapsulation rate=(1−free siRNA/total siRNA)×100%

### 3) Particle Size and Polydispersity

RDVs containing 1 μg siRNA are diluted to a final volume of 3 ml with 1×PBS. The particle size and polydispersity of the samples is measured by a dynamic light scattering method using ZetaPALS instrument (Brookhaven Instruments Corporation, Holtsville, N.Y.). The scattered intensity is measured with He—Ne laser at 25° C. with a scattering angle of 90°.

### 4) Zeta Potential Analysis

RDVs containing 1 μg siRNA are diluted to a final volume of 2 ml with milliQ H₂O. Electrophoretic mobility of samples is determined using ZetaPALS instrument (Brookhaven Instruments Corporation, Holtsville, N.Y.) with electrode and He—Ne laser as a light source. The Smoluchowski limit is assumed in the calculation of zeta potentials.

### 5) Lipid Analysis

Individual lipid concentrations are determined by Reverse Phase High-Performance Liquid Chromatography (RP-HPLC) using Waters 2695 Alliance system (Water Corporation, Milford Mass.) with a Corona charged aerosol detector (CAD) (ESA Biosciences, Inc, Chelmsford, Mass.). Individual lipids in RDVs are analyzed using a Agilent Zorbax SB-C18 (50×4.6 mm, 1.8 μn particle size) column with CAD at 60° C. The mobile phase is composed of A: 0.1% TFA in H₂O and B: 0.1% TFA in IPA. The gradient is 75% mobile phase A and 25% mobile phase B from time 0 to 0.10 min; 25% mobile phase A and 75% mobile phase B from 0.10 to 1.10 min; 25% mobile phase A and 75% mobile phase B from 1.10 to 5.60 min; 5% mobile phase A and 95% mobile phase B from 5.60 to 8.01 min; and 75% mobile phase A and 25% mobile phase B from 8.01 to 13 min with flow rate of 1 ml/min. The individual lipid concentration is determined by comparing to the standard curve with all the lipid components in the RDVs with a quadratic curve fit. The molar percentage of each lipid is calculated based on its molecular weight.

Utilizing the above described LNP process, specific LNPs with the following ratios were identified:

### Nominal Composition:

### Cationic Lipid/Cholesterol/PEG-DMG 60/38/2

### Cationic Lipid/Cholesterol/PEG-DMG 67.3/29/3.7.

Luc siRNA
(SEQ. ID. NO.: 1)
5′-iB-AUAAGGCUAUGAAGAGAUATT-iB 3′
(SEQ ID. NO.: 2)
3′-UUUAUUCCGAUACUUCUCUAU-5′
AUGC-Ribose
iB-Inverted deoxy abasic
UC-2′ Fluoro
AGT-2′ Deoxy
AGU-2′ OCH₃

### Example 1

### In Vivo Evaluation of Efficacy and Toxicity

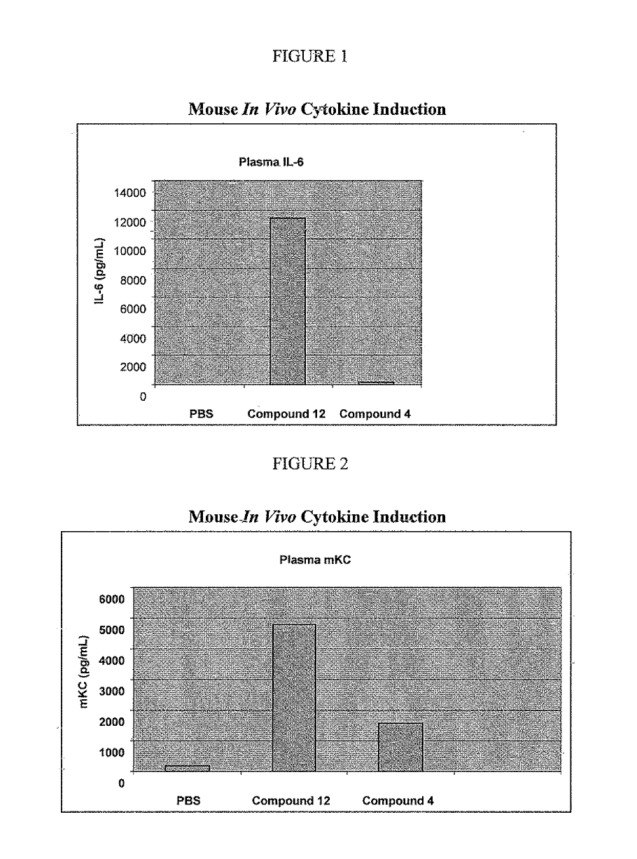

LNPs utilizing compound 4, in the nominal compositions described immediately above, were evaluated for in vivo efficacy and induction of inflammatory cytokines in a luciferase mouse model. The siRNA targets the mRNA transcript for the firefly (Photinus pyralis) luciferase gene (Accession #M15077). The primary sequence and chemical modification pattern of the luciferase siRNA is displayed above. The in vivo luciferase model employs a transgenic mouse in which the firefly luciferase coding sequence is present in all cells. ROSA26-LoxP-Stop-LoxP-Luc (LSL-Luc) transgenic mice licensed from the Dana Farber Cancer. Institute are induced to express the Luciferase gene by first removing the LSL sequence with a recombinant Ad-Cre virus (Vector Biolabs). Due to the organo-tropic nature of the virus, expression is limited to the liver when delivered via tail vein injection. Luciferase expression levels in liver are quantitated by measuring light output, using an IVIS imager (Xenogen) following administration of the luciferin substrate (Caliper Life Sciences). Pre-dose luminescence levels are measured prior to administration of the RDVs. Luciferin in PBS (15 mg/mL) is intraperitoneally (IP) injected in a volume of 150 uL. After a four minute incubation period mice are anesthetized with isoflurane and placed in the IVIS imager. The RDVs (containing siRNA) in PBS vehicle were tail vein injected n a volume of 0.2 mL. Final dose levels ranged from 0.3 to 3 mg/kg siRNA. PBS vehicle alone was dosed as a control. Three hours post dose, mice were bled retro-orbitally to obtain plasma for cytokine analysis. Mice were imaged 48 hours post dose using the method described above. Changes in luciferin light output directly correlate with luciferase mRNA levels and represent an indirect measure of luciferase siRNA activity. In vivo efficacy results are expressed as % inhibition of luminescence relative to pre-dose luminescence levels. Plasma cytokine levels were determined using the Searchlight multiplexed cytokine chemoluminescent array (Pierce/Thermo). Systemic administration of the luciferase siRNA RDVs decreased luciferase expression in a dose dependant manner. Greater efficacy was observed in mice dosed with compound 4 containing RDVs than with the RDV containing the octyl-CLinDMA cationic lipid, Compound 12, (Table 1). Compound 12 RDVs significantly increased mouse plasma levels of the cytokines IL-6 and mKC relative to the PBS control. However, administration of compound 4 produced minimal cytokine induction (FIGS. 1 & 2).

TABLE 1
Mouse In Vivo efficacy data. Average % Inhibition of
Bioluminescence by LNPs prepared from compound 4
compared against compound 12 at 3 mg Kg⁻¹

| Compound 4 | Compound 12 |
|---|---|
| 87 | 76 |

What is claimed is:

1. A cationic lipid of Formula A which is: 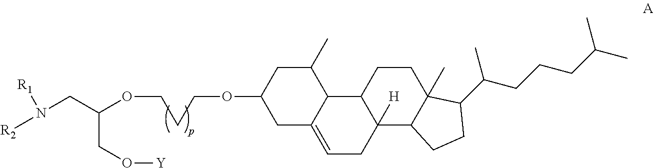
wherein:
p is 1 to 8;
R¹ and R² are independently selected from H, (C₁-C₁₀)alkyl, heterocyclyl, and a polyamine, wherein said heterocyclyl or polyamine is optionally substituted with one to three substituents selected from R³, or R¹ and R² can be taken together with the nitrogen to which they are attached to form a monocyclic heterocycle with 4-7 members optionally containing, in addition to the nitrogen, one or two additional heteroatoms selected from N, O and S, said monocyclic heterocycle optionally substituted with one to three substituents selected from R³;
R³ is independently selected from: halogen, OR⁴, SR⁴, CN, CO₂R⁴, CON(R⁴)₂;
R⁴ is independently selected from: H, (C₁-C₁₀)alkyl and aryl; and
Y is a (C₄-C₈)alkyl, (C₄-C₈)perfluoroalkyl, or a (C₄-C₈)alkenyl;
or any pharmaceutically acceptable salt or stereoisomer thereof.

2. A cationic lipid of Formula A according to claim 1, wherein:
p is 1 to 8; 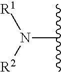
is selected from: 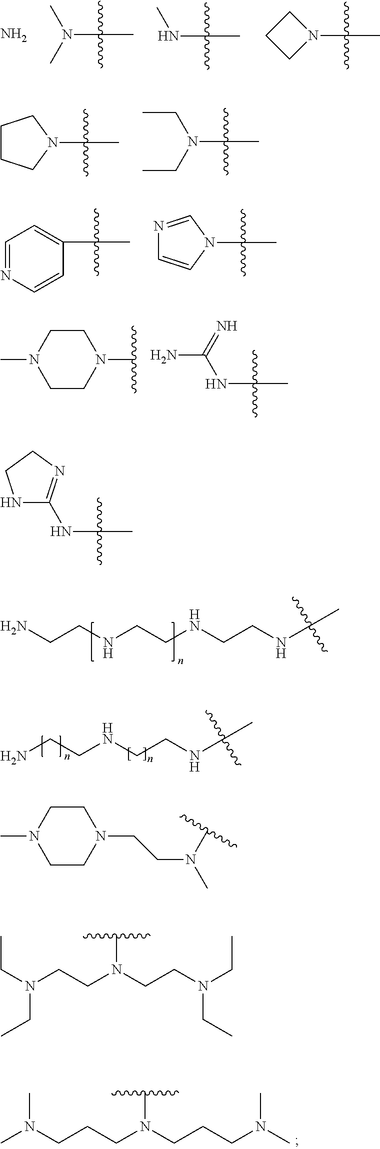
n is 1 to 10; and
Y is a (C₄-C₈)alkyl, (C₄-C₈)perfluoroalkyl, or a (C₄-C₈)alkenyl;
or any pharmaceutically acceptable salt or stereoisomer thereof.

3. A cationic lipid of Formula A according to claim 1 which is selected from:
(2S)-2-{4-[(3β)-cholest-5-en-3-yloxy]butoxy}-N,N-dimethyl-3-(octyloxy)propan-1-amine;
(2R)-2-{4-[(3β)-cholest-5-en-3-yloxy]butoxy}-N,N-dimethyl-3-(octyloxy)propan-1-amine;
(2R)-2-({6-[(3β)-cholest-5-en-3-yloxy]hexyl}oxy)-N,N-dimethyl-3-(octyloxy)propan-1-amine;
(2R)-2-({8-[(3β)-cholest-5-en-3-yloxy]octyl}oxy)-N,N-dimethyl-3-(octyloxy)propan-1-amine;
1-[(2R)-2-({8-[(3β)-cholest-5-en-3-yloxy]octyl}oxy)-3-(octyloxy)propyl]guanidine;
1-[(2R)-2-{4-[(3β)-cholest-5-en-3-yloxy]butoxy}-3-(octyloxy)propyl]guanidine;
(3β)-3-(4-{[(2R)-1-(octyloxy)-3-(pyrrolidinyl-1-yl)propan-2-yl]oxy}butoxy)cholest-5-ene; and
(3β)-3-[(8-{[(2S)-1-(octyloxy)-3-(pyrrolidinyl-1-yl)propan-2-yl]oxy}octyl)oxy]cholest-5-ene
or any pharmaceutically acceptable salt or stereoisomer thereof.

4. The use of a cationic lipid according to claim 1 for the preparation of lipid nanoparticles.

5. The use of a cationic lipid according to claim 1 as a component in a lipid nanoparticle for the delivery of oligonucleotides.

6. The use according to claim 5 wherein the oligonucleotides are siRNA or miRNA.

7. The use according to claim 5 wherein the oligonucleotides are siRNA.


SEQUENCE LISTING (2 sequences)

SEQ ID NO: 1 - DNA, 21 bp, Artificial Sequence
  Synthetic
auaaggcuaugaagagauatt

SEQ ID NO: 2 - RNA, 21 nt, Artificial Sequence
  Synthetic
uuuauuccgauacuucucuau